(19) United States
(12) Patent Application Publication
(10) Pub. No.: US 2004/0024273 A1
(43) Pub. Date: Feb. 5, 2004

(54) Method for the hydrogenation of aromatics by means of reactive distillation

(76) Inventors: Arnd Bottcher, Frankenthal (DE); Carsten Oost, Bad Durkheim (DE); Mathias Haake, Mannheim (DE)

Correspondence Address: Keil & Weinkauf, 1350 Connecticut Avenue NW, Washington, DC, 20036, US

(21) Appl. No.: 10/398,175

(22) PCT Filed: Oct. 9, 2001
(86) PCT No.: PCT/EP01/11650

(30) Foreign Application Priority Data
Oct. 13, 2000 (DE) 100507115

(51) Int. Cl. C07C 5/10

(52) U.S. Cl. 585/266; 585/268; 585/275

(57) ABSTRACT

In a process for hydrogenating unsubstituted monocyclic or polycyclic aromatics or monocyclic or polycyclic aromatics substituted by at least one alkyl group, amino group or hydroxyl group or a combination of two or more thereof to form the corresponding cycloaliphatics by means of gaseous hydrogen in the presence of at least one catalyst in a reaction column (4) in which the reactants are passed over the catalyst(s) (5) fixed in the reaction column (4), the cycloaliphatics are taken off at a side offtake (14) or from the bottom of the column (6) through a line (8) or at the side offtake (14) and from the bottom of the column (6) through a line (8).

The present invention relates to a process for hydrogenating monocyclic or polycyclic aromatics which may be substituted by at least one alkyl group, amino group or hydroxyl group or a combination or two or more thereof to give the corresponding cycloaliphatics. In particular, the present invention relates to a process for hydrogenating benzene to cyclohexane by means of reactive distillation in a reaction column in which the reactants are passed in countercurrent over the catalyst(s) fixed in the reaction column.

There are numerous processes for hydrogenating, for example, benzene to cyclohexane. These hydrogenation processes are predominantly carried out in the gas or liquid phase over particulate nickel and platinum catalysts (cf., for example, U.S. Pat. No. 3,597,489, GB 1 444 499 or GB 992 104). Typically, the major part of the benzene is hydrogenated to cyclohexane in a main reactor and the conversion into cyclohexane is subsequently completed in one or more after-reactors.

The strongly exothermic hydrogenation reaction is carried out with careful control of pressure, temperature and residence time in order to achieve complete conversion at a high selectivity. In particular, significant formation of methylcyclopentane, which is promoted by relatively high temperatures, has to be suppressed. Typical cyclohexane specifications require a residual benzene content of <100 ppm and a methylcyclopentane content of <200 ppm. The content of n-paraffins (n-hexane, n-pentane, etc.) is also critical. The formation of these undesirable compounds is likewise promoted by relatively high hydrogenation temperatures and, as in the case of methylcyclopentane, complicated separation operations are necessary to remove them from the cyclohexane produced. The removal can be carried out, for example, by extraction, rectification or by use of molecular sieves, as described in GB 1 341 057. The catalyst used for the hydrogenation also has a strong influence on the extent of formation of undesirable methylcyclopentane.

In view of this background, it is desirable to carry out the hydrogenation at the lowest temperatures possible. However, this is limited by the fact that, depending on the type of hydrogenation catalyst used, a hydrogenation activity of the catalyst sufficiently high for achieving economical space-time yields is attained only at relatively high temperatures.

The nickel and platinum catalysts used for the hydrogenation of benzene have a series of disadvantages. Nickel catalysts are very sensitive to sulfur-containing impurities in the benzene, so that it is necessary either to use very pure benzene for the hydrogenation or, as described in GB 1 104 275, to use a platinum catalyst which tolerates a higher sulfur content in the main reactor so as to protect the after-reactor which is charged with a nickel catalyst. Other possibilities are to dope the catalyst with rhenium (GB 1 155 539) or to use ion exchangers in the production of the catalyst (GB 1 144 499). However, the production of such catalysts is complicated and expensive. The hydrogenation can also be carried out over Raney nickel (U.S. Pat. No. 3,202,723), but a disadvantage is the ready combustibility of this catalyst. Homogeneous nickel catalysts can also be used for the hydrogenation (EP-A 0 668 257). However, these catalysts are very water-sensitive, so that the benzene used firstly has to be dried to a residual water content of <1 ppm in a drain column prior to the hydrogenation. A further disadvantage of the homogeneous catalyst is that it cannot be regenerated at justifiable cost.

Platinum catalysts have fewer disadvantages than nickel catalysts, but are much more expensive to produce. Both the use of platinum catalysts and the use of nickel catalysts require very high hydrogenation temperatures, which can lead to significant formation of undesirable by-products.

The hydrogenation of benzene to cyclohexane is not carried out industrially over ruthenium catalysts, but the patent literature refers to the use of ruthenium-containing catalysts for this application:

In SU 319 582, suspended Ru catalysts doped with Pd, Pt or Rh are used for preparing cyclohexane from benzene. However, the catalysts are very expensive because of the use of Pd, Pt or Rh. Furthermore, the work-up and recovery of suspension catalysts is complicated and expensive.

In SU 403 658, a Cr-doped ruthenium catalyst is used for preparing cyclohexane. The hydrogenation is carried out at 180° C., and a significant amount of undesirable by-products is generated.

U.S. Pat. No. 3,917,540 claims catalysts applied to Al₂O₃ as support material for preparing cyclohexane. These catalysts comprise, as active metal, a noble metal of transition group VIII of the Periodic Table, and also an alkali metal and technetium or rhenium. The Al₂O₃ supports are in the form of spheres, granules or the like. A disadvantage of such catalysts is that a selectivity of only 99.5% is achieved.

Finally, U.S. Pat. No. 3,244,644 describes ruthenium hydrogenation catalysts applied to η-Al₂O₃ as support material, which are said to be suitable for the hydrogenation of benzene. These catalysts are in the form of particles having maximum dimensions of 0.635 cm (¼ inch) and have an active metal content of at least 5%; the preparation of η-Al₂O₃ is complicated and expensive.

In addition to the above-described particulate catalysts or suspension catalysts, monolithic supported catalysts in the form of ordered packing provided with catalytically active layers which can be used for hydrogenation reactions are known from the prior art.

For example, EP-B 0 564 830 describes a monolithic supported catalyst which can comprise elements of group VIII of the Periodic Table as active components.

EP-A 0 803 488 discloses a process for the reaction, for example hydrogenation, of an aromatic compound bearing at least one hydroxyl group or amino group on an aromatic ring. The reaction is carried out in the presence of a catalyst comprising a homogeneous ruthenium compound which has been deposited in situ on a support, for example a monolith. The hydrogenation is carried out at pressures of more than 50 bar and temperatures of preferably from 150° C. to 220° C.

WO 96/27580 describes a process for hydrogenating unsaturated cyclic and polycyclic compounds by means of catalytic distillation, in which the reactor is operated at a pressure at which the reaction mixture boils under a low hydrogen partial pressure.

WO 98/09930 discloses a process for the selective hydrogenation of aromatic compounds in a mixed hydrocarbon stream by means of catalytic distillation in the presence of a catalyst.

In the processes of the two last-named publications, pressures of from 13.8 to 17.2 bar and temperatures of from 135 to 190° C. are necessary to achieve a satisfactory space-time yield. According to both publications, the desired product is always taken off or obtained at the top.

In all the processes described in the literature for the hydrogenation of aromatic compounds, the strongly exothermic hydrogenation reaction requires careful temperature and residence time control to achieve complete conversion at high selectivity. In particular, it is necessary to suppress any significant formation of methylcyclopentane, which is promoted by high temperatures. The by-products such as methylcyclopentane formed in the hydrogenation lead to contamination of the product in the abovementioned processes of the prior art. For this reason, the preparation of, for example, high-purity cyclohexane requires a subsequent distillation step, which is associated with capital costs.

It is an object of the present invention to provide an economical process for preparing cycloaliphatics by hydrogenation of the corresponding aromatics, in particular by hydrogenation of benzene to give cyclohexane, which makes it possible to obtain cycloaliphatics of high purity with high selectivity, in a high yield and under mild reaction conditions.

We have found that this object is achieved by a process for hydrogenating unsubstituted monocyclic or polycyclic aromatics or monocyclic or polycyclic aromatics substituted by at least one alkyl group, amino group or hydroxyl group or a combination of two or more thereof to form the corresponding cycloaliphatics by means of gaseous hydrogen in the presence of at least one catalyst in a reaction column in which the reactants are passed over the catalyst(s) fixed in the reaction column, wherein the cycloaliphatics are taken off at a side offtake or from the bottom of the column or at the side offtake and from the bottom of the column.

The reactants are preferably passed in countercurrent over the catalyst(s) fixed in the reaction column.

If the cycloaliphatics desired as product are taken off via a side offtake, the lower-boiling components (low boilers) are taken off at the top of the column. Correspondingly, the components having boiling points higher than that of the cycloaliphatic (high boilers) are obtained at the bottom of the column. Accordingly, the mode of operation is matched to the respective by-products which are present in aromatics or are formed during the reaction. For example, low boilers are taken off at the top and, correspondingly, high-boiling components are taken off from the bottom, while the cycloaliphatic is obtained via a side offtake.

If no high-boiling by-products or secondary components are present, the desired product is taken off from the bottom.

Of course, a mode of operation in which the cycloaliphatics are obtained as desired products via the side offtake and at the bottom of the column is also possible according to the present invention.

According to the present invention, whether the cycloaliphatics are obtained at the side offtake or at the bottom of the column is controlled by means of the reflux ratio in the column and/or the energy input into the column. At the side offtake, the product is preferably taken off in liquid form.

It has surprisingly been found that aromatics, nonlimiting examples of which are benzene, toluene, xylenes and aniline, can be hydrogenated selectively and at a high space-time yield to the corresponding cycloaliphatics by means of the process of the present invention at, compared to the processes of the prior art, significantly lower pressures and temperatures and that the cycloaliphatics are obtained in high purity in one apparatus.

In the process of the present invention, the hydrogenation is preferably carried out at a pressure of <20 bar and a temperature of <200° C.

In a particularly preferred embodiment, the hydrogenation is carried out at a pressure of <13 bar and a temperature of <150° C.

Even more preferably, the hydrogenation is carried out at a pressure in the range from 1 to 20 bar, preferably from 5 to 13 bar, and/or at a temperature in the range from 50 to 200° C., preferably from 80 to 150° C.

Since the system is in the boiling state, the temperature of the reaction mixture in the process of the present invention can be regulated in a simple manner by the pressure.

In the process of the present invention, the pressure is set so that the hydrogen partial pressure in the hydrogenation is in the range from 0.1 to 20 bar, preferably in the range from 5 to 13 bar.

In the process of the present invention, the catalytic hydrogenation is carried out over a heterogeneous catalyst in a reaction column; in principle, all catalysts suitable for this application can be used.

Specific examples are: shaped bodies made of catalytically active ion exchangers as described in Chem. Eng. Technol. 16 (1993), pages 279 to 289, which may be in the form of Raschig rings, saddles and other shapes known from distillation technology. A further example of catalytically active shaped bodies similar in configuration to internals in distillation technology are the KATAPAK catalysts and catalyst supports from Sulzer and the MULTIPAK catalysts produced by Montz. In terms of their geometry, they correspond to the cross-channel structures widespread in distillation technology, for example the types Sulzer BX, CY, DX, MELAPAK or Montz A3, BSH. Similar structures, but in the form of wire meshes which are additionally roughened, are disclosed in DE-A 19624130.8.

It is also possible to use catalysts, e.g. ion exchangers, which are embedded in pockets of wire meshes and rolled up into bales having a diameter of from about 0.2 to 0.6 m, with the height of such a bale being about 0.3 m. One or more of these bales is/are installed in the distillation column. Further information regarding such catalysts may be found in U.S. Pat. No. 4,215,011 and in Ind. Eng. Chem. Res. (1997), 36, pages 3821 to 3832, the relevant contents of which are hereby incorporated by reference into the present application.

It is also possible to use heterogeneous catalysts comprising active metals. Active metals which can be used are in principle all metals of transition group VIII of the Periodic Table. The active metal used is preferably platinum, rhodium, palladium, cobalt, nickel or ruthenium or a mixture of two or more thereof. Particular preference is given to using ruthenium as active metal.

Among the metals of transition groups I and VII of the Periodic Table, which can all likewise be used in principle, preference is given to using copper and/or ruthenium.

Particular preference is given to using ruthenium alone. An advantage of the use of ruthenium as hydrogenation metal is that, compared to the significantly more expensive hydrogenation metals platinum, palladium or rhodium, it enables considerable costs to be saved in catalyst production.

The ruthenium catalyst which is preferably used in the process of the present invention is placed in the column either in the form of a bed or as catalytically active distillation packing or in combinations of the two. The form of such a bed or distillation packing is already known to a person skilled in the art from the prior art.

Examples of metallic materials as support materials are pure metals such as iron, copper, nickel, silver, aluminum, zirconium, tantalum and titanium or alloys such as steels or stainless steels, e.g. nickel steel, chromium steel and/or molybdenum steel. It is also possible to use brass, phosphor bronze, Monel and/or nickel silver or combinations of two or more of the abovementioned materials.

Examples of ceramic materials are aluminum oxide (Al₂O₃), silicon dioxide (SiO₂), zirconium dioxide (ZrO₂), cordierite and/or steatite.

Examples of synthetic support materials are plastics such as polyamides, polyesters, polyethers, polyvinyls, polyolefins such as polyethylene, polypropylene, polytetrafluoroethylene, polyketones, polyether ketones, polyether sulfones, epoxy resins, aldehyde resins, urea- and/or melamine-aldehyde resins. It is also possible to use carbon as support.

Preference is given to using structured supports in the form of woven meshes, knitted meshes, woven carbon fiber fabrics or carbon fiber felts or woven or knitted polymer fabrics. Possible woven wire meshes are woven meshes made of weavable metal wires such as iron, spring steel, brass, phosphor bronze, pure nickel, Monel, aluminum, silver, nickel silver, nickel, chromium-nickel, chromium steel, stainless, acid-resistant and high-temperature-resistant chromium-nickel steels and also titanium.

It is likewise possible to use woven meshes made of inorganic materials, for example woven meshes made of ceramic materials such as Al₂O₃ and/or SiO₂.

Synthetic wires and woven fabrics made of polymers are also able to be used according to an embodiment of the invention.

Monoliths made of woven packing are particularly preferred since they withstand high cross-sectional throughputs of gas and liquid and display only insignificant abrasion. In a further particularly preferred embodiment, use is made of metallic, structured supports or monoliths made of stainless steel whose surface has preferably been roughened by heating in air and subsequent cooling. These properties are displayed, in particular, by stainless steels in the case of which an alloy constituent accumulates at the surface above a specific demixing temperature and forms a strongly adhering rough oxidic surface layer by oxidation in the presence of oxygen. Such an alloy constituent can be, for example, aluminum or chromium from which a surface layer of Al₂O₃ or Cr₂O₃ is formed. Examples of stainless steels are those of material numbers 1.4767, 1.4401, 1.4301, 2.4610, 1.4765, 1.4847 and 1.4571. These steels are preferably thermally roughened by heating in air at from 400 to 1100° C. for a period of from 1 hour to 20 hours and subsequent cooling to room temperature.

In a preferred embodiment, the heterogeneous catalyst is a ruthenium-coated woven mesh which at the same time acts as distillation packing. In a still more preferred embodiment of the process of the present invention, the distillation packing comprises ruthenium-coating metal threads, with particular preference being given to using stainless steel number 1.4301 or 1.4767.

As is known to a person skilled in the art from the prior art, it is also possible to use a promoter or a plurality of promoters for the catalyst. The promoters can be, for example, alkali metals and/or alkaline earth metals such as lithium, sodium, potassium, rubidium, cesium, magnesium, calcium, strontium and barium, coinage metals such as copper, silver and/or gold, zinc, tin, bismuth, antimony, molybdenum, tungsten and/or other promoters such as sulfur and/or selenium.

Before application of the active metals and any promoters, the structured or monolithic supports may be coated with one, two or more oxides. This can be achieved physically, for example by sputtering. Here, elements and/or element compounds are sputtered onto the support material in an oxidizing atmosphere under high-vacuum conditions. Suitable elements are, for example, titanium, silicon, zirconium, aluminum and zinc. Further details may be found in EP-B 0 564 830, the relevant contents of which are hereby fully incorporated by reference into the present application.

In some cases, it is also possible to use high-vacuum vapor deposition (for example electron beam), which is likewise described in EP-B 0 564 830.

The structured supports can, either before or after application of the active metals and promoters, be shaped or rolled up, for example by means of a tooth roller, to produce a monolithic catalyst element.

The catalysts used according to the present invention can be produced industrially by application of at least one metal of transition group VIII of the Periodic Table and, if desired, at least one promoter to one of the above-described supports.

The application of the active metals and any promoters to the above-described supports can be carried out by vaporizing the active metals under reduced pressure and condensing them continuously onto the support. Another possibility is to apply the active metals to the supports by impregnation with solutions comprising the active metals and any desired promoters. A further possibility is to apply the active metals and any promoters to the supports by chemical methods, for example chemical vapor deposition (CVD).

The catalysts produced in this way can be used directly or can be heat treated and/or calcined before use, and can be used either in a prereduced state or in an unreduced state.

If desired, the support is pretreated before application of the active metals and any promoters. Pretreatment is advantageous, for example, when adhesion of the active components to the support is to be improved. Examples of pretreatments are coating the support with adhesion promoters and roughening by mechanical (e.g. grinding, sandblasting) or thermal means such as heating, generally in air, or plasma etching.

In a preferred embodiment, the present invention provides a process of this type in which the catalyst comprises, as active metal, at least one metal of transition group VIII of the Periodic Table either alone or together with at least one metal of transition group I or VII of the Periodic Table applied to a support which has a mean pore diameter of at least 50 nm and a BET surface area of not more than 30 m²/g, with the amount of active metal being from 0.01 to 30% by weight, based on the total weight of the catalyst (catalyst 1). More preferably, the mean pore diameter of the support in this catalyst is at least 0.1 μm and the BET surface area is not more than 15 m²/g (catalyst 1a).

The present invention further provides a process of this type in which the catalyst comprises, as active metal, at least one metal of transition group VIII of the Periodic Table either alone or together with at least one metal of transition group I or VII of the Periodic Table in an amount of from 0.01 to 30% by weight, based on the total weight of the catalyst, applied to a support, where from 10 to 50% of the pore volume of the support is made up by macropores having a pore diameter in the range from 50 nm to 10,000 nm and from 50 to 90% of the pore volume of the support is made up by mesopores having a pore diameter in the range from 2 to 50 nm, where the sum of the pore volumes adds up to 100% (catalyst 2).

As supports, it is in principle possible to use all supports which have only macropores and also those which have both macropores and mesopores and/or micropores.

For the purposes of the present invention, the terms “macropores” and “mesopores” are used as defined in Pure Appl. Chem., 45, p. 79 (1976), namely as pores whose diameter is above 50 nm (macropores) or whose diameter is in the range from 2 nm to 50 nm (mesopores). “Micropores” are likewise defined as in the above reference and the term refers to pores having a diameter of <2 nm.

The active metal content is generally from about 0.01 to about 30% by weight, preferably from about 0.01 to about 5% by weight and in particular from about 0.1 to about 5% by weight, in each case based on the total weight of the catalyst used. The contents preferably used in the preferred catalysts 1 and 2 described below are indicated individually in the discussion of these catalysts.

The preferred catalysts 1 and 2 will now be described in detail. The description is based by way of example on the use of ruthenium as active metal. The details provided below are also applicable to the other active metals which can be used, as defined herein.

Catalyst 1

The catalysts 1 used according to the present invention can be produced industrially by applying at least one metal of transition group VIII of the Periodic Table and optionally at least one metal of transition group I or VII of the Periodic Table to a suitable support.

The application can be carried out by steeping the support in aqueous metal salt solutions, e.g. aqueous ruthenium salt solutions, by spraying appropriate metal salt solutions onto the support or by other suitable methods. Suitable metal salts of elements of transition groups I, VII and VIII of the Periodic Table are the nitrates, nitrosyl nitrates, halides, carbonates, carboxylates, acetylacetonates, chloro complexes, nitrito complexes or amine complexes of the corresponding metals, with preference being given to the nitrates and nitrosyl nitrates.

In the case of catalysts in which further metals in addition to the metal of transition group VIII of the Periodic Table are applied as active metal to the support, the metal salts or metal salt solutions can be applied simultaneously or in succession.

The supports which have been coated or impregnated with the metal salt solution are subsequently dried, preferably at from 100 to 150° C., and if desired calcined at from 200 to 600° C., preferably from 350 to 450° C. In the case of separate impregnation, the catalyst is dried after each impregnation step and if desired calcined, as described above. The order in which the active components are applied can be chosen without restriction.

The coated and dried and if desired calcined supports are subsequently activated by treatment in a gas stream comprising free hydrogen at from about 30 to about 600° C., preferably from about 150 to about 450° C. The gas stream preferably consists of from 50 to 100% by volume of H₂ and from 0 to 50% by volume of N₂.

The metal salt solution or solutions is/are applied to the support in such a manner that the total active metal content, in each case based on the total weight of the catalyst, is from about 0.01 to about 30% by weight, preferably from about 0.01 to about 5% by weight, more preferably from about 0.01 to about 1% by weight and in particular from about 0.05 to about 1% by weight.

The total metal surface area on the catalyst 1 is preferably from about 0.01 to about 10 m²/g, more preferably from about 0.05 to about 5 m²/g and in particular from about 0.05 to about 3 m²/g, of the catalyst. The metal surface area is determined by means of the chemisorbtion method described by J. Lemaitre et al. in “Characterization of Heterogeneous Catalysts”, Ed. Francis Delanney, Marcel Dekker, New York 1984, pp. 310-324.

In the catalyst 1 used according to the present invention, the ratio of the surface areas of the active metal/metals and of the catalyst support is preferably less than about 0.05, with the lower limit being about 0.0005.

Support materials which can be used for producing the catalysts used according to the present invention are ones which are macroporous and have a mean pore diameter of at least about 50 nm, preferably at least about 100 nm, in particular at least about 500 nm, and whose BET surface area is not more than about 30 m²/g, preferably not more than about 15 m²/g, more preferably not more than about 10 m²/g, in particular not more than about 5 m²/g and more preferably not more than about 3 m²/g. The mean pore diameter of the support is preferably from about 100 nm to about 200 μm, more preferably from about 500 nm to about 50 μm. The BET surface area of the support is preferably from about 0.2 to about 15 m²/g, more preferably from about 0.5 to about 10 m²/g, in particular from about 0.5 to about 5 m²/g and more preferably from about 0.5 to about 3 m²/g.

The surface area of the support is determined by the BET method by N₂ adsorption, in particular in accordance with DIN 66131. The mean pore diameter and the pore size distribution are determined by Hg porosimetry, in particular in accordance with DIN 66133.

The pore size distribution of the support is preferably approximately bimodal, with the pore diameter distribution having maxima at about 600 nm and about 20 μm in the bimodal distribution representing a specific embodiment of the invention.

Greater preference is given to a support which has a surface area of 1.75 m²/g and this bimodal distribution of the pore diameter. The pore volume of this preferred support is preferably about 0.53 ml/g.

Examples of macroporous support material which can be used are macroporous activated carbon, silicon carbide, aluminum oxide, silicon dioxide, titanium dioxide, zirconium dioxide, magnesium oxide, zinc oxide or mixtures of two or more thereof, with particular preference being given to using aluminum oxide and zirconium dioxide.

Further details regarding catalyst 1 and its production may be found in DE-A 196 24 484.6, the relevant contents of which are hereby fully incorporated by reference into the present application.

Support materials which can be used for producing the catalysts 1a which are used according to the present invention and represent a preferred embodiment of catalyst 1 are ones which are macroporous and have a mean pore diameter of at least 0.1 μm, preferably at least 0.5 μm, and a surface area of not more than 15 m²/g, preferably not more than 10 m²/g, particularly preferably not more than 5 m²/g, in particular not more than 3 m²/g. The mean pore diameter of the support used there is preferably in a range from 0.1 to 200 μm, in particular from 0.5 to 50 μm. The surface area of the support is preferably from 0.2 to 15 m²/g, particularly preferably from 0.5 to 10 m²/g, in particular from 0.5 to 5 m²/g, especially from 0.5 to 3 m²/g, of the support. This catalyst, too, has the above-described bimodality of the pore diameter distribution with analogous distributions and the corresponding preferred pore volumes. Further details regarding catalyst 1a may be found in DE-A 196 04 791.9, the relevant contents of which are hereby incorporated by reference into the present application.

Catalyst 2

The catalysts 2 used according to the present invention comprise one or more metals of transition group VIII of the Periodic Table as active component(s) on a support, as defined herein. Preference is given to using ruthenium, palladium and/or rhodium as active component(s).

The catalysts 2 used according to the present invention can be produced industrially by applying at least one active metal of transition group VIII of the Periodic Table, preferably ruthenium, and optionally at least one metal of transition group I or VII of the Periodic Table to a suitable support. The application can be carried out by steeping the support in aqueous metal salt solutions, e.g. aqueous ruthenium salt solutions, by spraying appropriate metal salt solutions onto the support or by other suitable methods. Suitable metal salts for the preparation of the metal salt solutions are the nitrates, nitrosyl nitrates, halides, carbonates, carboxylates, acetylacetonates, chloro complexes, nitrito complexes or amine complexes of the corresponding metals, with preference being given to the nitrates and nitrosyl nitrates.

In the case of catalysts in which a plurality of active metals have been applied to the support, the metal salts or metal salt solutions can be applied simultaneously or in succession.

The supports which have been coated or impregnated with the metal salt solution are subsequently dried, preferably at from 100 to 150° C. If desired, the supports can be calcined at from 200 to 600° C., preferably from 350 to 450° C. The coated supports are subsequently activated by treatment in a gas stream comprising free hydrogen at from 30 to 600° C., preferably from 100 to 450° C. and in particular from 100 to 300° C. The gas stream preferably consists of from 50 to 100% by volume of H₂ and from 0 to 50% by volume of N₂.

If a plurality of active metals are applied to the supports and the application is carried out in succession, the support can be dried at from 100 to 150° C. and optionally calcined at from 200 to 600° C. after each application or impregnation. The order in which the metal salt solution is applied can be chosen without restriction.

The metal salt solution is applied to the support or supports in such an amount that the active metal content is from 0.01 to 30% by weight, preferably from 0.01 to 10% by weight, more preferably from 0.01 to 5% by weight, in particular from 0.3 to 1% by weight, based on the total weight of the catalyst.

The total metal surface area on the catalyst is preferably from 0.01 to 10 m²/g, particularly preferably from 0.05 to 5 m²/g and more preferably from 0.05 to 3 m²/g, of the catalyst. The metal surface area is determined by the chemisorbtion method described in J. Lemaitre et al., “Characterization of Heterogeneous Catalysts”, Ed. Francis Delanney, Marcel Dekker, New York (1984), pp. 310-324.

In the catalyst 2 used according to the present invention, the ratio of the surface areas of the active metal or metals and of the catalyst support is less than about 0.3, preferably less than about 0.1 and in particular about 0.05 or less, with the lower limit being about 0.0005.

The support materials which can be used for producing the catalysts 2 used according to the present invention possess macropores and mesopores.

Here, the supports which can be used according to the present invention have a pore distribution in which from about 5 to about 50%, preferably from about 10 to about 45%, more preferably from about 10 to about 30% and in particular from about 15 to about 25%, of the pore volume is made up by macropores having pore diameters in the range from about 50 nm to about 10,000 nm, and from about 50 to about 95%, preferably from about 55 to about 90%, more preferably from about 70 to about 90% and in particular from about 75 to about 85%, of the pore volume is made up by mesopores having a pore diameter of from about 2 to about 50 nm, where in each case the sum of the pore volumes adds up to 100%.

The total pore volume of the supports used according to the present invention is from about 0.05 to 1.5 cm³/g, preferably from 0.1 to 1.2 cm³/g and in particular from about 0.3 to 1.0 cm³/g. The mean pore diameter of the supports used according to the present invention is from about 5 to 20 nm, preferably from about 8 to about 15 nm and in particular from about 9 to about 12 nm.

The surface area of the support is preferably from about 50 to about 500 m²/g, more preferably from about 200 to about 350 m²/g and in particular from about 250 to about 300 m²/g, of the support.

The surface area of the support is determined by the BET method by N₂ adsorption, in particular in accordance with DIN 66131. The mean pore diameter and the size distribution are determined by Hg porosimetry, in particular in accordance with DIN 66133.

Although it is in principle possible to use all support materials known in catalyst production which have the above-defined pore size distribution, preference is given to using activated carbon, silicon carbide, aluminum oxide, silicon dioxide, titanium dioxide, zirconium dioxide, magnesium oxide, zinc oxide or mixtures thereof, more preferably aluminum oxide and zirconium dioxide.

Further details regarding catalyst 2 may be found in DE-A 196 24 485.4, the relevant contents of which are hereby fully incorporated by reference into the present application.

Further details regarding the catalysts used in the process of the present invention, their structure and production may be found in DE 199 17 051.7, the relevant contents of which are hereby fully incorporated by reference into the present application.

The low-boiling by-products formed in the reaction are distilled off via the top during the reactive distillation, possibly as azeotrope with the starting materials, and discharged from the reaction system. In an analogous manner, any high-boiling by-products formed are separated off via the bottom.

The energy liberated in the strongly exothermic reaction is utilized for distillation.

Both benzene and its substituted derivatives such as toluene or xylene can be converted into the corresponding saturated hydrocarbons by means of the process of the present invention.

In the process of the present invention, it is in principle possible to use all monocyclic or polycyclic aromatics which are either unsubstituted or substituted by at least one alkyl group, amino group or hydroxyl group or a combination of two or more thereof, either singly or as mixtures of two or more thereof, preferably singly. The length of the alkyl group is subject to no particular restrictions, but the alkyl groups are generally C₁-C₃₀-, preferably C₁-C18-, in particular C₁-C₄-alkyl groups.

Furthermore, aromatic compounds in which at least one hydroxyl group and preferably also at least one substituted or unsubstituted C₁-C₁₀-alkyl radical and/or alkoxy radical are bound to an aromatic ring can be hydrogenated according to the present invention to form the corresponding cycloaliphatic compounds, with it also being possible to use mixtures of two or more of these compounds.

The aromatic compounds can be monocyclic or polycyclic aromatic compounds. The aromatic compounds contain at least one hydroxyl group which is bound to an aromatic ring; the simplest compound of this type is phenol. The aromatic compounds preferably have one hydroxyl group per aromatic ring. The aromatic compounds can be substituted on the aromatic ring or rings by one or more alkyl and/or alkoxy radicals, preferably C₁-C₁₀-alkyl and/or alkoxy radicals, particularly preferably C₁-C₁₀-alkyl radicals, in particular methyl, ethyl, propyl, isopropyl, butyl, isobutyl, tert-butyl radicals; among alkoxy radicals, preference is given to C₁-C₈-alkoxy radicals such as methoxy, ethoxy, propoxy, isopropoxy, butoxy, isobutoxy, tert-butoxy radicals. The aromatic ring or rings and also the alkyl and alkoxy radicals may be substituted by halogen atoms, in particular fluorine atoms, or bear other suitable inert substitutents.

The compounds which can be hydrogenated according to the present invention preferably contain at least one, more preferably from 1 to 4, in particular 1, C₁-C₁₀-alkyl radical which may be located on the same aromatic ring as the hydroxyl group or groups. Preferred compounds are (mono)alkylphenols, in which the alkyl radical can be in the o-, m- or p-position relative to the hydroxyl group. Particular preference is given to trans-alkylphenols, also referred to as 4-alkylphenols, where the alkyl radical preferably has from 1 to 10 carbon atoms and is particularly preferably a tert-butyl radical. Preference is given to 4-tert-butylphenol. Polycyclic aromatic compounds which can be used according to the present invention are, for example, β-naphthol and α-naphthol.

The aromatic compounds in which at least one hydroxyl group and preferably also at least one substituted or unsubstituted C₁-C₁₀-alkyl radical and/or alkoxy radical is/are bound to an aromatic ring can also have a plurality of aromatic rings which are linked via an alkylene radical, preferably a methylene group. The linking alkylene group, preferably methylene group, can bear one or more alkyl substituents which may be C₁-C₂₀-alkyl radicals and are preferably C₁-C₁₀-alkyl radicals, particularly preferably methyl, ethyl, propyl, isopropyl, butyl or tert-butyl radicals.

Each of the aromatic rings may have at least one hydroxyl group bound to it. Examples of such compounds are bisphenols which are linked in the 4 position via an alkylene radical, preferably a methylene radical.

In the process of the present invention, particular preference is given to hydrogenating a phenol substituted by a C₁-C₁₀-alkyl radical, preferably C₁-C₆-alkyl radical, where the alkyl radical may be substituted by an aromatic radical, or mixtures of two or more of these compounds.

In a further, preferred embodiment of this process, p-tert-butylphenol, bis(p-hydroxyphenyl)dimethylmethane or a mixture thereof is hydrogenated.

The process of the present invention can also be used to hydrogenate aromatic compounds in which at least one amino group is bound to an aromatic ring to form the corresponding cycloaliphatic compounds, with it also being possible to use mixtures of two or more of these compounds. The aromatic compounds can be monocyclic or polycyclic aromatic compounds. The aromatic compounds contain at least one amino group bound to an aromatic ring. The aromatic compounds are preferably aromatic amines or diamines. The aromatic compounds can be substituted on the aromatic ring or rings or on the amino group by one or more alkyl and/or alkoxy radicals, preferably C₁-C₂₀-alkyl radicals, in particular methyl, ethyl, methoxy, ethoxy, propoxy, isopropoxy, butoxy, isobutoxy, tert-butoxy radicals. The aromatic ring or rings and also the alkyl and alkoxy radicals may be substituted by halogen atoms, in particular fluorine atoms, or bear other suitable inert substitutents.

The aromatic compound in which at least one amino group is bound to an aromatic ring may also have a plurality of aromatic rings which are linked via an alkylene group, preferably a methylene group. The linking alkylene group, preferably methylene group, can have one or more alkyl substituents which may be C₁-C₂₀-alkyl radicals, preferably C₁-C₁₀-alkyl radicals, particularly preferably methyl, ethyl, propyl, isopropyl, butyl, sec-butyl or tert-butyl radicals.

The amino group bound to the aromatic ring can likewise be substituted by one or two of the above-described alkyl radicals.

Particularly preferred compounds are aniline, naphthylamine, diaminobenzenes, diaminotoluenes and bis-p-aminophenylmethane or mixtures thereof.

Specifically, the present process is particularly preferably used for hydrogenating the following aromatics: benzene, toluene, xylenes, cumene, diphenylmethane, tribenzenes, tetrabenzenes, pentabenzenes and hexabenzenes, triphenylmethane, alkyl-substituted naphthalenes, naphthalene, alkyl-substituted anthracenes, anthracene, alkyl-substituted tetralins and tetralin, and also aniline. Preference is given to hydrogenating benzene to cyclohexane in the present process.

Although the hydrogenation of the aromatics can be carried out with the hydrogen-containing gas and the liquid aromatic or aromatics being passed in cocurrent through a column, the hydrogenation of the present invention is preferably carried out with the hydrogen-containing gas being passed through a column equipped with one of the above-described catalysts in countercurrent to the liquid aromatic or aromatics. Here, the liquid phase can be passed through the column from the top downward and the gaseous phase from the bottom upward. The hydrogenation is preferably carried out in two or more stages. The catalyst described in the present application is used in at least one stage.

As hydrogenation gases, it is possible to use any gases which comprise free hydrogen and contain no harmful amounts of catalyst poisons, for example CO. For example, off-gases from reformers can be used. Preference is given to using pure hydrogen as hydrogenation gas.

The hydrogenation of the present invention can be carried out in the absence or presence of a solvent or diluent, i.e. it is not necessary to carry out the hydrogenation in solution.

As solvent or diluent, it is possible to use any suitable solvent or diluent. The choice is not critical as long as the solvent or diluent used is able to form a homogeneous solution with the aromatic to be hydrogenated.

The amount of solvent or diluent used is not subject to any particular restrictions and can be chosen freely according to requirements, but preference is given to amounts which lead to a 10-70% strength by weight solution of the aromatic to be hydrogenated.

When using a solvent in the process of the present invention, the product formed in the hydrogenation, i.e. the respective cycloaliphatic(s), is used as preferred solvent(s), if desired in addition to other solvents or diluents. In this case, part of the product formed in the process can be mixed into the aromatic still to be hydrogenated.

The present, novel process has numerous advantages compared to processes of the prior art. The reactive distillation combines chemical reactions and fractional distillation of the starting materials and products in one apparatus. This offers process engineering advantages in respect of the way in which the reaction is carried out and reduces energy consumption. In addition, capital cost savings compared to carrying out reaction and distillation in separate apparatuses can be made.

In addition, the process of the present invention enables the aromatics to be hydrogenated selectively and in a high space-time yield to give the corresponding cycloaliphatics at significantly lower pressures and temperatures than those described in the prior art. The catalysts have a high activity even at relatively low pressures and temperatures. The cycloaliphatics are obtained in highly pure form. Even at low pressures, cycloaliphatics can be obtained in a high space-time yield. Furthermore, the hydrogenation can be carried out with excellent selectivity without the addition of auxiliary chemicals.

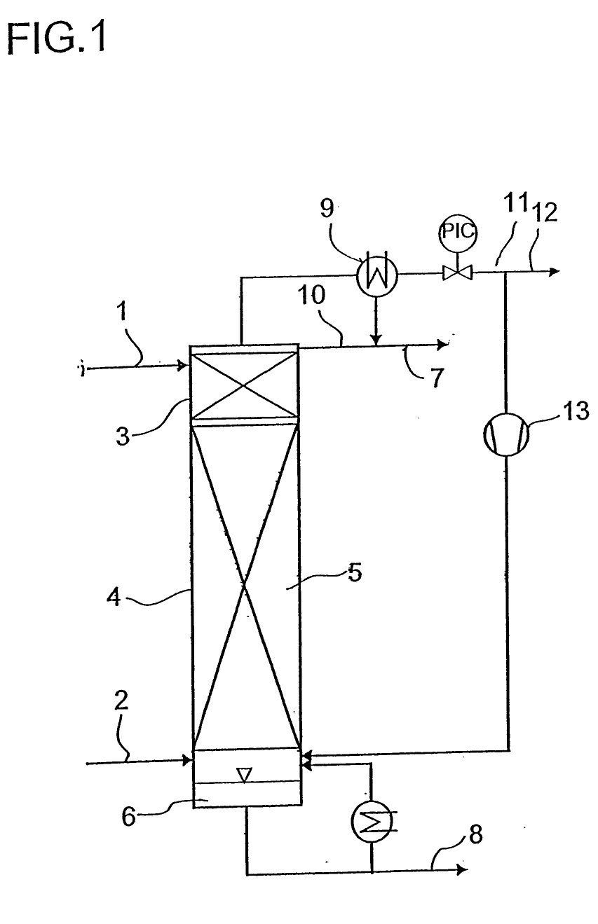

FIG. 1 shows a simplified flow diagram of a distillation apparatus for carrying out the process of the present invention in which the cycloaliphatic is obtained at the bottom of the column.

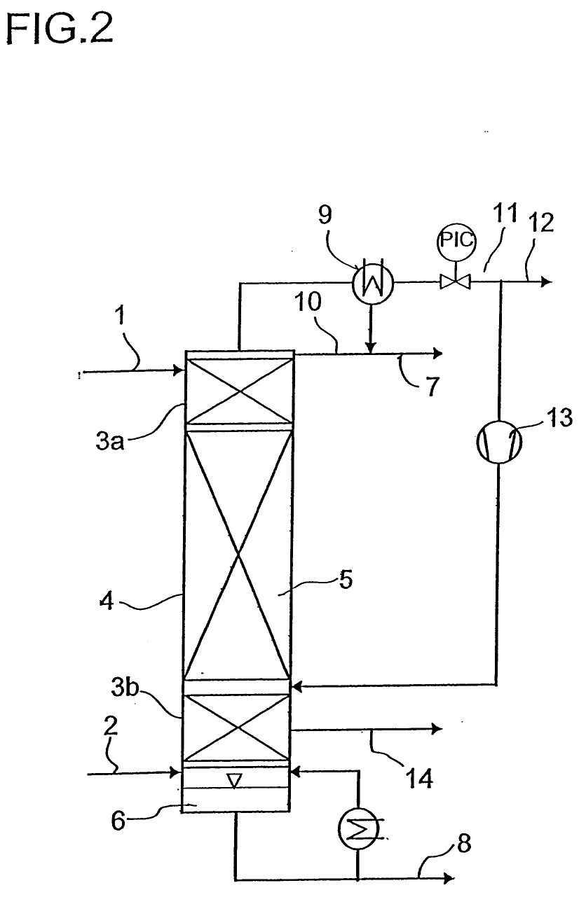

FIG. 2 shows such a simplified flow diagram of a distillation apparatus for carrying out the process of the present invention in which the cycloaliphatic is taken off via a side offtake.

The figures will now be discussed in more detail for, by way of example, the preparation of cyclohexane from benzene.

In the process of the present invention as shown in FIG. 1, the reaction is carried out by means of reactive distillation over a heterogeneous catalyst 5, as described above, in a reaction column 4. The feed point 1 for benzene opens into the upper part 3 of the reaction column 4 and the feed point 2 for hydrogen opens into the lower part of the reaction column 4. In this way, the reactants flow in countercurrent through the reaction column 4. Benzene reacts over the heterogeneous catalyst 5 to form cyclohexane with simultaneous distillation. Cyclohexane is the low boiler of the system, distilled into the bottom of the column 6 and is discharged through the line 8.

Since benzene and cyclohexane form a low-boiling azeotrope, the concentration profile in the process of the present invention is set so that no benzene is present in the bottom of the column 6 and a region of high concentration of benzene or benzene/cyclohexane is present on the heterogeneous catalyst 5.

The by-products formed in the reaction are low boilers and are, possibly as azeotrope with benzene or cyclohexane, condensed out in the top condenser 9. The predominant part of the benzene-containing stream taken off at the top is returned as runback 10 to the reaction column 4 and a small part 7 of the stream taken off at the top containing the by-products is discharged. Furthermore, low-boiling impurities present in the benzene can likewise be separated off in a simple manner before the reaction zone with the heterogeneous catalyst 5 and be discharged via the part 7 of the stream from the top of the column.

Unreacted hydrogen 11 obtained at the top of the reaction column 4 leaves the reaction column 4 together with the relatively low-boiling components, and is, optionally after discharge of a substream 12, recirculated by means of a compressor 13 to the bottom 6 of the reaction column 4.

When the process of the present invention is carried out by means of an apparatus as shown in FIG. 2, the desired cycloaliphatic product, here cyclohexane, is taken off via a side offtake 14 located in the lower part of the column 3b. In this embodiment, the high boilers are obtained via line 8 at the bottom of the column 6. In contrast to FIG. 1, the upper part of the column in FIG. 2 is denoted by 3a; otherwise the reference numerals in FIG. 2 correspond to those of FIG. 1.

The invention is illustrated by the following examples.

### EXAMPLE

Catalyst A:

This catalyst is a commercially available catalyst comprising 0.5% of ruthenium on Al₂O₃ spheres having mesopores and macropores corresponding to catalyst 2 according to the present invention.

Catalyst B:

The catalytic packing of this catalyst was produced from woven metal mesh which had previously been coated with ruthenium. The production method is described in EP-A 0 564 830, the relevant contents of which are hereby incorporated by reference into the present application.

Carrying Out the Process

The experimental apparatus comprised a heatable 2 liter stainless steel reaction flask which was fitted with a stirrer and a superposed distillation column (length: 1 m; diameter 50 mm) made up of two column sections. The lower part (0.5 m) of the distillation column was in one experiment provided with the above-described catalyst A and in another experiment with the catalyst B, while the upper region of the distillation column was provided with a Montz B1-750 distillation packing. The benzene was metered into the uppermost section of the distillation column by means of a pump. The water was metered into the distillation flask. In this way, countercurrent flow of the reactants of the catalyst was achieved.

The hydrogen and by-products formed were separated off via the reaction column and condensed in a partial condenser. The condensate flowed over a runback divider into a reservoir. The remaining off-gas stream was passed through a cold trap and subsequently through a gas meter to measure the volume.

The apparatus was equipped with a pressure regulator and designed for a system pressure of 20 bar.

All inflowing and outflowing streams were continually measured and recorded during the time of the experiment, so that a time-dependent mass balance was possible.

As an alternative, a comparative experiment in the downflow mode was carried out using the same apparatus.

The experimental conditions and experimental results are shown in Table 1.

TABLE 1
Hydrogenation of benzene to cyclohexane

| | | Ben- | Outflow | Out- | | | | Benzene | Cyclo- | | Benzene | Cyclo- | |
|---|---|---|---|---|---|---|---|---|---|---|---|---|---|
| | | zene | at the | flow of | Pressure | T | | in | hexane in | BP in | in | hexane in | BP in |
| Exper- | | feed | bottom | distillate | abs | bottom | T top | distillate | distillate | distillate | bottoms | bottoms | bottoms |
| iment | Column | [g/h] | [g/h] | [g/h] | [bar] | [° C.] | [° C.] | [%] | [%] | [%] | [%] | [%] | [%] |
| Benz1 | Var1_RD | 92 | 85 | 7 | 6 | 157 | 118 | 75.3 | 41.4 | 1.3 | <100 ppm | 100.0 | <100 ppm |
| Benz2 | Var2_DM | 100 | 100 | 0 | 6 | 157 | 25 | — | — | — | 18.8 | 80.5 | 0.71 |
| Benz3 | Var3_RD | 100 | 100 | 5 | 6 | 151 | 118 | 56.2 | 40.2 | 1.1 | <100 ppm | 100.0 | <100 ppm |
| Benz4 | Var2_DM | 100 | 100 | 0 | 6 | 151 | 25 | — | — | — | 12.0 | 88.0 | <100 ppm |

Var1: 1st section: Catalyst bed of catalyst 1
2nd section: Montz mesh packing B1-750
Var2: 1st section: Mesh packing with catalyst 2
2nd section: Montz mesh packing B1-750
RED = Reactive distillation
DM = Downflow mode

We claim:

1. A process for hydrogenating unsubstituted monocyclic or polycyclic aromatics or monocyclic or polycyclic aromatics substituted by at least one alkyl group, amino group or hydroxyl group or a combination of two or more thereof to form the corresponding cycloaliphatics by means of gaseous hydrogen in the presence of at least one catalyst in a reaction column in which the reactants are passed over the catalyst(s) fixed in the reaction column, wherein the cycloaliphatics are taken off at a side offtake or from the bottom of the column or at the side offtake and from the bottom of the column.

2. A process as claimed in claim 1, wherein the reactants are passed in countercurrent over the catalyst(s) fixed in the reaction column.

3. A process as claimed in claim 1 or 2, wherein the hydrogenation is carried out at a pressure of <20 bar and a temperature of <200° C.

4. A process as claimed in any of claims 1 to 3, wherein the hydrogenation is carried out at a pressure of <13 bar and a temperature of <150° C.

5. A process as claimed in any of claims 1 to 4, wherein a heterogeneous catalyst is used.

6. A process as claimed in claim 5, wherein a ruthenium catalyst is used.

7. A process as claimed in claim 5 or claim 6, wherein a ruthenium catalyst in the form of a bed in the reaction column and/or in the form of distillation packing in the column, preferably in the form of ruthenium-coated distillation packing comprising inorganic or organic threads, is used.

8. A process as claimed in any of claims 1 to 7, wherein the hydrogenation is carried out at a pressure in the range from 1 to 20 bar, preferably from 5 to 13 bar, and/or at a temperature in the range from 50 to 200° C., preferably from 80 to 150° C.

9. A process as claimed in any of claims 1 to 8, wherein the hydrogen partial pressure during the hydrogenation is in the range from 0.1 to 20 bar, preferably from 5 to 13 bar.

10. A process as claimed in any of claims 1 to 9, wherein undesired by-products are separated off via the top during the reactive distillation.

11. A process as claimed in any of claims 1 to 10, wherein cyclohexane is prepared from benzene, methylcyclohexane is prepared from toluene, dimethylcyclohexane is prepared from xylene, or cyclohexylamine is prepared from aniline.
